(19) United States
(12) Patent Application Publication
(10) Pub. No.: US 2009/0070889 A1
(43) Pub. Date: Mar. 12, 2009

(54) MOUSE MODEL COMPRISING AN ENGRAFTED HUMAN SKIN HAVING HYPERSENSITIVITY TO UV-LIGHT

(75) Inventors: Marcela Nechaevsky Del Rio, Madrid (ES); Marta Garcia Diez, Madrid (ES); Jose Luis Jorcano, Madrid (ES); Carlos Ramon Trullas Cabanas, Viladecavalls (ES); Alvaro Meana Infiesta, Gijon (ES)

Correspondence Address: NORRIS, MCLAUGHLIN & MARCUS, PA, 875 THIRD AVENUE, 18TH FLOOR, NEW YORK, NY, 10022, US

(73) Assignee: CIEMAT, Madrid (ES)

(21) Appl. No.: 12/203,457

(22) Filed: Sep. 3, 2008

Related U.S. Application Data
(63) Continuation of application No. PCT/EP2007/001801, filed on Mar. 2, 2007.

(30) Foreign Application Priority Data
Mar. 3, 2006 (EP) 06 004 354.4

(51) Int. Cl. A01K 67/027

(52) U.S. Cl. 800/10; 800/9

(57) ABSTRACT

The present invention relates to a humanized, non-human mammal model, preferably a humanized mouse model, with an engrafted portion of human skin having hyper-patient sensitivity to ultra violet (UV) light, and a method for preparing such non-human mammal model and its use for studying acute and long term effects on human skin by exposure to UV light and for testing topically or systematically applied or administered substances for their capability to prevent, repair and/or cure damages of such exposed human skin. This mouse model comprises an engrafted portion of human skin based on skin cells of a human Xeroderma pigmentosum (XP) or Gorlin's Syndrome (GS) patient. XP and GS are rare human autosomal disorders characterized clinically by hypersensitivity to UV light, especially to UVB rays, and high predisposition for developing skin cancers on sunlight exposed skin areas.

The present invention relates to a skin-humanized, non-human mammal model, preferably a skin-humanized mouse model, with an engrafted portion of human skin with hypersensitivity to ultra violet (UV) light, a method for preparing such non-human mammal model and its use for studying acute and long term effects on human skin by exposure to UV light and for testing topically or systemically applied or administered substances for their capability to prevent, repair and/or cure damages of such exposed human skin.

It is known that excessive exposure to solar ultra violet (UV) irradiation causes severe acute damages to the skin, especially human skin, including erythema, suntan, sunburn, immunosupression as well as long term damages such as photoaging and skin cancer like basal cell carcinoma, squamous cell carcinoma or melanoma skin cancer.

It is also known that the study of the in vivo biological effects of UV irradiation on human volunteers is restricted for ethical and technical reasons.

Therefore there is a need for a reliable animal model that allows not only studying acute and so called long term effects on human skin by exposure to UV light but also carrying out these studies after only a reasonable time period since the average time period between exposure to UV light and the occurrence of the related effects is shortened dramatically.

Moreover, there is also a need for a reliable animal model that allows not only testing substances to prevent, repair and/or cure damages of human skin due to UV irradiation but also carrying out these tests after only a reasonable time period has expired since the exposure as mentioned above. This means that there is a need for a reliable animal model that allows studying the effects of UV-light exposure in a kind of quick motion manner.

The present invention solves the above mentioned needs by providing a skin-humanized, non-human mammal model comprising an engrafted portion of human skin with hypersensitivity to ultra violet (UV) light.

Preferably the non-human mammal is a rodent, more preferably a mouse. The inventive non-human mammal is a not-transgenic mammal.

In order to fulfil the above mentioned task the inventive, skin-humanized, non-human mammal model comprises an engrafted portion of human skin based on skin cells of a human Xeroderma pigmentosum (XP) or Gorlin's Syndrome (GS) patient. XP and GS are rare human autosomal disorders characterized clinically by hypersensitivity to UV light and high predisposition for developing skin cancers on sunlight exposed skin areas.

Human skin derived from skin cells of XP or GS patients shows a hypersensitivity to UV rays, especially to UVB rays.

In order to prepare the inventive, skin-humanized, non-human mammal model with an engrafted portion of human skin with hypersensitivity to UV light

1. a skin equivalent is bioengineered by using skin cells isolated form a human XP or GS patient,

2. a portion of this skin equivalent is grafted on the designated part, preferably the back of the non-human mammal like a mouse, preferably orthotopically and

3. this grafted portion of skin equivalent is protected with the removed, devitalized skin of the non-human mammal until the human skin has regenerated on the non-human mammal.

The grafting procedure has to be performed under sterile conditions and the skin humanized animal models have to be housed under pathogen free conditions.

In order to bioengineer the skin equivalent, skin biopsies are taken form a human XP or GS patient. Human keratinocytes hypersensitive to UV light and human dermal fibroblasts are obtained form these skin biopsies by a double enzymatic digestion (trypsin and collagenase respectively). These primary keratinocytes are cultivated in a known manner. The dermal fibroblasts also derived from the biopsy are also cultured as known.

As dermal component of the bioengineered skin which will be grafted either a fibrin matrix populated with the above mentioned cultured live human dermal fibroblasts or a human plasma based matrix with these fibroblasts embedded therein is used.

Such a fibrin matrix can be prepared by adding said human dermal fibroblasts to a fibrinogen solution (derived from cryoprecipitates of human blood) which is solidified after the addition of known agent like Ca Cl₂ and thrombin to a fibrin matrix gel.

Details of preparing such a dermal matrix are disclosed in the publication “Large surface of cultured human epithelium obtained on a dermal matrix based on live fibroblast-containing fibrin gels” by Meana A, Iglesias J, Del Rio M, et al. in BURNS 1998; 24: 621. The respective disclosure for the preparation of such dermal matrix and the growing of human keratinocytes is hereby incorporated by reference and forms part of the present disclosure.

It is also possible to use a dermal matrix based on human plasma optionally from the same human being from whom the skin biopsies are taken. According to such a dermal matrix the human fibroblasts derived from skin cell biopsy are embedded in the clotted human plasma based matrix.

A preparation of such a dermal matrix is disclosed in the publication “Human plasma as a dermal scaffold for the generation of a completely autologous bioengineered skin” by Sara G. Llames, Marcela Del Rio, Fernando Larcher, et al. in “TRANSPLANTATION”, Vol. 77, 350-355, No. 3, Feb. 15, 2004.

The respective disclosure for the preparation of such dermal matrix is hereby incorporated by reference and forms part of the present disclosure.

Either on this plasma-based matrix serving as a three-dimensional scaffold or on the above mentioned fibrin matrix populated with the fibroblasts derived from a XP or GS patient the cultivated keratinocytes also obtained from skin biopsies of this XP or GS patient are seeded to form the epidermal layer of the artificial skin a portion of which is to be grafted on the non-human mammal, preferably on a mouse. The culture medium is preferably the same as that used for the primary keratinocytes cultures on feeder layer. Usually the culturing is continued until at least the keratinocytes' growing on the matrix gel reaches confluence.

The thus prepared bioengineered (artificial) skin is ready to be grafted in a non-human mammal. Therefore a portion of the bioengineered skin is preferably fixed to a gauze with an appropriate glue and detached form the culture flash. For grafting the bioengineered skin equivalent on the non-human mammal, preferably on a mouse, more preferably on a nude mouse, the bioengineered skin portion is preferably placed orthotopically on the non-human mammal, preferably on its back, where a wound matching with the skin portion has to be created. During the take process the portion of skin equivalent is preferably protected with the devitalized skin removed from the non-human mammal for being replaced by the bioengineered skin.

This biologic bandage is preferably kept in place until the human skin becomes visible. The whole procedure has to be carried out under sterile conditions and the skin-humanized, non-human mammals have to be housed under pathogen free conditions.

A further object of the present invention is therefore a method for preparing the inventive skin-humanized, non-human mammal model as described in detail above.

Since the inventive, skin-humanized, non-human mammal model has an engrafted portion of human skin with a hypersensitivity to UV light it is an ideal model to study the effects of the exposure of a human skin to UV light. Moreover, since these effects appear on the grafted skin after a considerable shorter exposure to UV light than on normal human skin because of the hypersensitivity of the grafted human skin to UV light the studies can be performed in a far less time consuming manner. This means that not only the acute damages like erythema, suntan, sunburn and/or immunosupression can be studied with the inventive model in a very efficient and reliable way but also the damages known as long term damages in consequence of skin exposure to UV light such as photoaging or skin cancer of various kinds. These damages already appear on the human UV sensitive skin of the inventive model only after a fraction of the usual time between exposure to UV light and appearance of skin damages has passed.

A person skilled in the art knows how the effects of an exposure to UV light, specially UVB light, like reaction of the epidermal architecture, changes in keratinocyta proliferation-differentiation and/or DNA damages can be determined.

Therefore it is a further object of the present invention to provide a method for studying the effects such as those mentioned above on a human skin because of exposure to irradiation with UV-light, especially UVB light by using the inventive, skin-humanized, non-human mammal model, preferably an inventive skin-humanized mouse model.

The inventive animal model is also an effective and reliable model for testing the capability of a topically applied substance to prevent and/or repair and/or cure the acute effects and/or effects known as long term effects mentioned above like damages on a human skin exposed to UV irradiation by using the inventive model. Accordingly such a method for testing the capability of a topically applied substance, preferably in form of a topical formulation such as a creme, gel, lotion or body milk to prevent, repair and/or cure the above mentioned effects on human skin as a in consequence of UV, especially UVB irradiation by using the inventive skin-humanized model, especially by applying such substance respectively topical formulation on the grafted human skin portion of the inventive model is a further object of the present invention.

With the inventive skin-humanized model is also possible to test the capability of a substance which is administered to the inventive model systemically to prevent, to repair and/or cure the acute effects and/or effects known as long term effects on a human skin by exposure to UV irradiation, especially UVB irradiation. Therefore such method for testing the capability of a substance to prevent, repair and/or cure the above mentioned effects by systemical activity in the inventive, skin-humanized, non-human mammal model, preferably mouse model, is a further aspect of the present invention.

Preparation of skin-humanized mice (comprising a grafted human skin portion with hypersensitivity to UV light)

1. Cell Culture
Ultra UV sensitive human keratinocytes were obtained by taking a skin biopsy from a Xeroderma pigmentosum (XP) patient and by enzymatic digestion. Primary keratinocytes were cultivated on a feeder layer of lethally irradiated 3T3 cells. Human dermal fibroblasts were also derived form this skin biopsy and cultured in DMEM containing 10% fetal calf serum (FCS). Cells were cultured at 37° C. in a humid atmosphere containing 5% CO₂. The culture medium was changed every 2 days.

2. Fibrin-Based Artificial Skin Preparation
A fibrin matrix populated with the above obtained human live fibroblasts was used as the dermal component of the artificial skin. The fibrin matrix was prepared as follows: 3 ml of fibrinogen solution (from cryoprecipitates of human blood donors) were added to 12 ml of DMEM with 10% FCS containing 5×10⁵ dermal fibroblasts cultured as described in 1. and 500 IU of bovine aprotinin (Trasylol, Bayer). Immediately afterward 1 ml 0.025 mM Ca Cl₂ (Sigma, St. Louis, Mo.) with 11 IU of bovine thrombin (Sigma) was added. Finally, the mixture was placed on polycarbonate inserts (4 μm porous) in a 6-well culture plate (Transwell, Coastar, Cambridge, Mass.) and allowed to solidify at 37° C. for 2 h. Human keratinocytes cultivated as described in 1. were then seeded on the fibrin matrix to form the epidermal layer of the artificial skin. Organotypic cultures were grown submerged up to keratinocyte confluence, then fed only through the lower chamber. Cultures were maintained at the air-liquid interface for 7 days to enhance stratification and differentiation of the epithelium upon grafting.

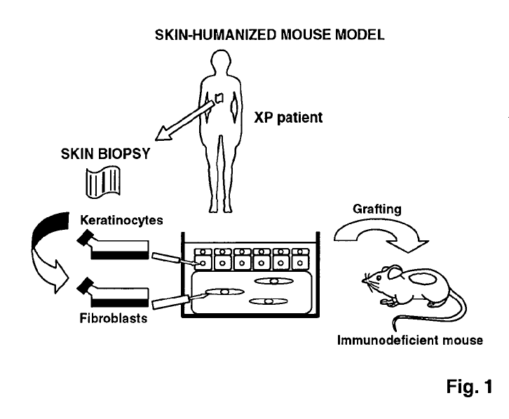

3. Grafting of the Bioengineered Skin Prepared in 2.
The bioengineered skin prepared in 2. was manually detached form the transwell and placed orthopically on backs of NOD/SCID or nude mice. The mice were shaved and aseptically cleansed before. Full thickness 12 mm circular wounds were then created on the dorsum of each mouse to match skin equivalent to be grafted. Mouse skin removed to generate the wound was de-vitalized by three repeated cycles of freezing and thawing and used as a biological bandage, fixed with sutures and covered with NewSkin (Medtech, Jackson, Wy.) to protect and hold the bioengineered skin in place during the take process. Dead mouse skin was sloughed off, generally within 7 days after grafting, and regenerated human skin became visible. Grafting was performed under sterile conditions and mice were housed in pathogen-free conditions for the duration of the experiment. Mice were housed in individually ventilated type II cages, with 25 air changes per hour and 10 Kg gamma-irradiated soft wood pellets as bedding.
FIG. 1 shows the preparation of the skin-humanized mouse model described above.

4. Study of the Skin Damages after a Single UVB Irradiation
The skin-humanized mice were exposed to a single UVB irradiation which UVB irradiation was performed using Philips TL20W/12 fluorescent tubes. Acute photodamages were evaluated 24 h after irradiation.
To study the DNA damage induced by UVB light, general skin morphology and sunburn cell formation were monitored by classical histology. In addition, immunostaining was performed using monoclonal antibodies against cyclobutane pyrimidine dimers and p53 to detect DNA lesions and p53 induction after UVB irradiation, respectively. Immunostainings were performed on deparaffinized sections using the mouse monoclonal antibody H3 (10). p53 protein was revealed also on deparaffinized sections using the anti-human p53 antibody (clone D07 Dako 4).
The number of p53-positive keratinocytes per millimeter epidermis was counted in at least five randomly selected fields from each slide (from each animal, in at least 5 animals from each group: irradiated and non-irradiated). Mean was significantly different between irradiated and non-irradiated skin-humanized mice using a Student's t-test where p≦0.001 (75.1±13.0 positive keratinocytes in the irradiated skin vs 0.7±0.2 positive keratinocytes in the non-irradiated skin).
It was found that after 24 h of irradiation with UV-B light the p53 induction was approximately 100-fold higher in irradiated mice compared to the p53 induction in non-irradiated mice.

1. A skin-humanized, non-human mammal model comprising an engrafted portion of human skin with hypersensitivity to ultra violet (UV) light.

2. A non-human mammal model as claimed in claim 1, wherein the mammal is a rodent.

3. A non-human mammal model as claimed in claim 1, wherein the mammal is a mouse.

4. A non-human mammal model as claimed in claim 1, wherein the mammal is a not-transgenic mammal.

5. A non-human mammal model as claimed in claim 1, wherein the mammal is an immunodeficient mammal.

6. A non-human mammal model as claimed in claim 1, wherein the portion of human skin has been regenerated after grafting a bioengineered skin equivalent on the non-human mammal.

7. A non-human mammal model as claimed in claim 1, wherein the portion of human skin is based on skin cells of a Xeroderma pigmentosum (XP) or Gorlin's Syndrome (GS) patient.

8. A non-human mammal model as claimed in claim 6, wherein at least the keratinocytes and fibroblasts of the bioengineered skin equivalent are derived from a skin biopsy of a XP or GS patient.

9. A non-human mammal model as claimed in claim 1, wherein the engrafted portion of human skin is a human skin with hypersensitivity to UV rays.

10. A method for preparing a skin-humanized, non-human mammal model with an engrafted portion of human skin with hypersensitivity to ultra violet (UV) light, as claimed in claim 1, comprising
a. bioengineering a skin equivalent of skin cells isolated from a human Xeroderma pigmentosum (XP) or Gorlin's Syndrome (GS) patient,
b. grafting a portion of the bioengineered skin equivalent on the designated part of the non-human mammal which matches this skin equivalent, and
c. protecting this grafted skin equivalent with devitalized skin removed from the non-human mammal until the human skin has regenerated.

11. A method as claimed in claim 10, wherein said bioengineering the skin equivalent comprises utilizing at least the keratinocytes and dermal fibroblasts obtained from the isolated skin cells of a XP or GS patient.

12. A method as claimed in claim 10, wherein said bioengineering the skin equivalent comprises preparing a fibrin matrix populated with said fibroblasts as the artificial dermis and seeding with said keratinocytes to form the epidermal layer of the artificial skin equivalent which is to be grafted on the non-human mammal.

13. A method for studying the effects of a human skin's exposure to irradiation with UV light comprising irradiating the skin-humanized, non-human mammal model as claimed in claim 1 with UV light and studying the effects of such UV light on the skin-humanized, non-human mammal model.

14. A method as claimed in claim 13, wherein the UV light is UVB light.

15. A method as claimed in claim 13, wherein the effects are acute effects selected from erytherma, suntan, sunburn and/or immunosupression.

16. A method as claimed in claim 13, wherein the effects are long term effects selected from photoaging and/or skin cancer.

17. A method for testing the capability of a substance after topical application to prevent and/or repair and/or cure the acute effects and/or long term effects of a human skin exposure to UV irradiation comprising irradiating the skin-humanized, non-human mammal model as claimed in claim 1 and determining the capability of said substance after topical application to prevent and/or repair and/or cure acute effects and/or long term effects of such irradiating.

18. A method as claimed in claim 17, wherein the substance is tested as one component of a topical formulation selected from cremes, gels, lotions or body milks.

19. A method for testing the capability of a substance after systemical administration to prevent, repair and/or cure acute effects and/or long term effects of a human skin exposure to UV irradiation comprising irradiating the skin-humanized, non-human mammal model as claimed in claim 1 and determining the capability of said substance after systemical administration to prevent, repair and/or cure acute effects and/or long term effects of such irradiating.

20. A method as claimed in claim 19, wherein said determining comprises determining the capability of a substance to prevent, to repair and/or cure photoaging and/or skin cancer by systemical administration of said substance to the skin-humanized, non-human mammal model before, during or after exposure to UV irradiation.
